(19) United States
(12) Patent Application Publication
(10) Pub. No.: US 2002/0068057 A1
(43) Pub. Date: Jun. 6, 2002

(54) TREATMENT OF AUTOIMMUNE AND INFLAMMATORY DISORDERS

(76) Inventors: MARC FELDMANN, LONDON (GB); RAVINDER N. MAINI, LONDON (GB); RICHARD O. WILLIAMS, LONDON (GB)

Correspondence Address: DAVID E BROOK, HAMILTON BROOK SMITH AND REYNOLDS, TWO MILITIA DRIVE, LEXINGTON, MA, 02173

(21) Appl. No.: 08/617,737

(22) PCT Filed: Mar. 10, 1994
(86) PCT No.: PCT/GB94/00462

(51) Int. Cl. A61K 38/16; A61K 38/00; A61K 39/395; A61K 39/40; A61K 39/42

(52) U.S. Cl. 424/133.1; 424/141.1; 424/152.1; 424/172.1; 514/8; 514/12

(57) ABSTRACT

A method for treating autoimmune or inflammatory diseases, through the administration of a CD4+ T cell inhibiting agent, such as anti-CD4 antibody or cyclosporin A, in conjunction with or sequentially to a TNF antagonist, such as anti-TNF antibody or soluble TNF receptor, is disclosed. The method can be used to aid in therapy for humans and other mammals with a wide variety of autoimmune or inflammatory diseases.

### BACKGROUND OF THE INVENTION

The nature of autoantigens responsible for autoimmune disorders is not known, nor is the action which triggers the autoimmune response. One popular theory involves the similarity of a viral protein to a self antigen, which results in autoreactive T cells or B cells recognizing a self antigen. Whereas B-lymphocytes produce antibodies, thymus-derived or “T-cells” are associated with cell-mediated immune functions. T-cella recognize antigens presented on the surface of cells and carry out their functions with these “antigen-presenting” cells.

Various markers have been used to define human T cell populations. CD4 is a non-polymorphic surface glycoprotein receptor with partial sequence identity to immunoglobulins. CD4 receptors define distinct subsets of mature peripheral T cells. In general, CD4 T cells expressing helper or regulatory functions interact with B cells in immune responses, while T calls expressing the CD8 surface antigen function as cytotoxic T cells and have regulatory effects on immune responses. Since T-cell receptors are the pathway through which stimuli augment or modulate T-cell responses, they present a potential target for immunological intervention.

Of the cellular interactions, that of CD4+ T calls with antigen presenting cells (APC) lies at the root of the immune response. Many aspects of the autoimmune response are essentially similar to that of normal immune responses. Thus CD4+ autoantigen reactive T cells are restimulated by APC expressing class II with autoantigen peptides in the binding groove. In certain human diseases the evidence that this occurs has been provided: in Graves' disease of the thyroid, in vivo activated T cells are present in the glands that are removed for refractory disease, and many of these cells after cloning can be shown to recognize autologous thyrocytes (as APC) not extrinsically supplied with any antigen, or APC supplied with the thyroid specific antigens thyroid peroxidase or thyroglobulin (Londei, M. et al., Science 228: 85-89 (1985); Dayan, C. M. et al., Proc. Natl. Acad. Sci. USA 88: 7415-7419 (1991)). Similarly, in rheumatoid arthritis (RA), in vivo activated T cells recognizing collagen type II have been isolated from joints of an RA patient in three consecutive operations during the course of three years (Londei, M. et al., Proc. Natl. Acad. Sci. 86: 636-640 (1989)). In other human diseases displaying autoimmune characteristics, CD4+ T cells from the blood have been cloned, including CD4+ T cells recognizing the acetylcholine receptor in myasthenia gravis (Hohlfeld, R. et al., Nature 310: 224-246 (1984)); myelin basic protein in multiple sclerosis (Hafler, D. A. et al., J. Immunol. 139: 68-72 (1987)); or islet cell membranes in insulin dependent diabetes mellitus (De Berardinis, P. et al., Lancet II: 823-824 (1988); Kontiainen, S. et al., Autoimmunity 8: 193-197 (1991)).

Factors other than CD4 also influence cellular immune response. The cytokine tumor necrosis factor-α (TNFα; also termed cachectin) has multiple effects on inflammation, tissue damage, immune response and cell trafficking into lesions, and thus plays a role in the pathogenesis of inflammatory joint diseases, including rheumatoid arthritis (Brennan, F. M. et al., Lancet 11, 244-247 (1989); Feldmann, M. et al., Ann. Rheumatic Dis. 51: 480-486 (l990)). TNFα is a protein secreted primarily by monocytes and macrophages in response to endotoxin or other stimuli as a soluble homotrimer of 17 kD protein subunits (Smith, R. A. et al., J. Biol. Chem. 262: 6951-6954 (1987)). A membrane-bound 26 kD precursor form of TNF has also been described (Kriegler, M. et al., Cell 53: 45-53 (1988). The expression of the gene encoding TNFα is not limited to cells of the monocyte/macrophage family: TMF is also produced by CD4+and CD8+ peripheral blood T lymphocytes, and by various cultured T and B cell lines (Cuturi, M. C. et al., J. Exp. Med. 165: (1581 (1987); Sung, S.-S. J. et al., J. Exp. Med. 168: 1539 (1988); Turner, M. et al., Eur. J. Immunol. 17: 1807-1814 (1987)). Recent evidence implicates TNF in the autoimmune pathologies and graft versus host pathology (Piguet, P.-F. et al., J. Exp. Med. 166; 1280 (1987).

Because of the multiple factors involved in autoimmune and inflammatory disorders, a great need exists for better therapies for autoimmune and inflammatory diseases.

### SUMMARY OF THE INVENTION

The current invention pertains to the discovery that combination therapy, involving the use of a CD4+ T cell inhibiting agent in conjunction with a TNF antagonist, produces markedly superior results than the use of each agent alone in the treatment of autoimmune or inflammatory disease, particularly in rheumatoid arthritis. CD4+ T cell inhibiting agents include agents which block, diminish, inhibit, or interfere with the activation of CD4+ T cells or the interaction of CD4+ T cells with antigen presenting cells (APC), such as antibodies to T cells or to their receptors; antibodies to APC or to their receptors; and other appropriate peptides or small molecules. TNF antagonists include agents which block, diminish, inhibit, or interfere with TNF activity, TNF receptors, or TNF synthesis, such as anti-TNF antibodies; soluble TNF receptors; and other appropriate peptides or small molecules.

In one embodiment of the current invention, anti-CD4 antibodies are administered in conjunction (either simultaneously or sequentially) with anti-TNF antibodies. In another embodiment of the current invention, anti-CD4 antibodies are administered in conjunction with soluble TNF receptor, such as a TNF receptor/TgG fusion protein. In a third embodiment of the current invention, cyclosporin is administered in conjunction with anti-TNF antibody. The combination therapy can utilize any CD4+ T cell inhibiting agent in conjunction with any TNF antagonist, including multiple CD4+ T cell inhibiting agents in conjunction with multiple TNF antagonists. Combination therapy can also utilize inflammatory mediators other than TNF antagonists, in conjunction with CD4+ T cell inhibiting agents.

The CD4+ T cell inhibiting agent and TNF antagonist can be administered together with a pharmaceutically acceptable vehicle; administration can be in the form of a single dose, or a series of doses separated by intervals of days or weeks.

The benefits of combination therapy with CD4+ T call inhibiting agents and TNF antagonists include improved results in comparison with the effects of treatment with each therapeutic modality separately. In addition, lower dosages can be used to provide the same reduction of the immune and inflammatory response, thus increasing the therapeutic window between a therapeutic and a toxic effect. Lower doses may also result in lower financial costs to the patient, and potentially fewer side effects.

### BRIEF DESCRIPTION OF THE FIGURES

FIG. 1 contains a set of graphs, individually labelled as FIG. 1A and FIG. 1B, from an experiment which illustrates the suppression of arthritis as assessed by clinical score (FIG. 1A) and pawswelling measurements (FIG. 1B) after the administration of 50 μg anti-TNF (hamster TN3.19.2) and 200 μg anti-CD4 to DSA/1 male mice. Open squares=control; diamonds=anti-CD4; triangles=anti-TNF; closed squares=anti-CD4/anti-TNF.

FIG. 2 contains a set of graphs, individually labelled as FIG. 2A, FIG. 2B, FIG. 2C, and FIG. 2D, from a second experiment which illustrates the potentiation of anti-CD4 with low dose (50 μg) anti-TNF or high dose (300 μg) anti-TNF on clinical score and pawswelling measurements. FIG. 2A: clinical score with low-dose anti-TNF; FIG. 2B: clinical score with high-dose anti-TNF; FIG. 2C: pawswelling with low-dose anti-TNF; FIG. 2D: pawswelling with high-dose anti-TNF. Open squares=control; diamonds=anti-CD4; triangles=anti-TNF; closed squares=anti-CD4/anti-TNF.

FIG. 3 is a graph illustrating the suppression of arthritis as assessed by pawswelling measurements after the administration of 250 μg cyclosporin A, 50 μg anti-TNF antibody, and a combination of 250 μg cyclosporin A and 50 μg anti-TNF antibody to DBA/1 mice. Open squares=control; diamonds=cyclosporin A; triangles=anti-TNF; closed squares=cyclosporin A/anti-TNF.

### DETAILED DESCRIPTION OF THE INVENTION

The present invention concerns the treatment of autoimmune or inflammatory diseases, such as rheumatoid arthritis, through the administration of a CD4+ T cell inhibiting agent in conjunction with a TNF antagonist. The invention also encompasses the use of multiple CD4+ T cell inhibiting agents in conjunction with multiple TNF antagonists. The term “CD4+ T cell inhibiting agent”, as used herein, refers to an agent which blocks, diminishes, inhibits, or interferes with the activation of CD4+ T cells or the interaction of CD4+ T cells with antigen presenting cells (APC). CD4+ T cell inhibiting agents include antibodies to T cells or to their receptors, such as anti-CD4, anti-CD28, anti-CD52 (e.g., CAMPATH-1H) and anti-IL-2R; antibodies to APC or to their receptors, such as anti-class II, anti-ICAM-1, anti-LFA-3, and anti-LFA-1; peptides and small molecules blocking the T cell/APC interaction, including those which block the HLA class II groove, or block signal transduction in T-cell activation, such as cyclosporins, particularly cyclosporin A, or FK-506; and antibodies to B cells including CD5+ B cells, such as CD19, 20, 21, 23 and BB/7 or B1, ligands for CD28, B cells including CD5+ B cells are considered to be an important type of APC in disease processes (Plater-Zyberk, C. et al., Ann. N.Y. Acad. Sci. 651; 540-555 (1992)), and thus anti-B cell antibodies can be particularly useful in the current invention.

The term “TNF antagonist”, as used herein, refers to an agent which blocks, diminishes, inhibits, or interferes with TNF activity, TNF synthesis, or TNF receptors, such as anti-TNF antibody; soluble TNF receptor (monomeric receptor and/or fusion proteins comprising the receptor, such as receptor/IgG fusion proteins, etc.); and other appropriate peptides or small molecules, such as pentoxyfilline or other phosphodiesterase inhibitors, and thalidomide.

Inflammatory mediators other than TNF antagonists can also be used instead of or in addition to TNF antagonists in the current invention. In rheumatoid joint cell cultures, Brennan et al. (Lancet 11, 244-247 (1989)) have shown that blocking TNF results in down-regulation of IL-1 production, and down-regulation of the pro-inflammatory cytokine GM-CSF (Haworth et al., E.J.I. 21:2575-2579 (1991); Brennan et al., in preparation). Unpublished data indicates that anti-TNF also blocks IL-6 production. These cytokine “networks” or “hierarchies” also operate in vivo; rheumatoid arthritis patients treated with anti-TNF antibody reduced their serum IL-6 levels, as well as levels of IL-6 dependent acute phase proteins such as C reactive protein, in the weeks following treatment (Elliott, M. J. et al., Arthritis & Rheumatism 36:1681-1690 (1993)). Since the pro-inflammatory mediators TNF, IL-1, GM-CSF, IL-6 and IL-8 are part of the same network or hierarchy, blocking any of these could have comparable effects and thus can be used as the inflammatory mediators of the current invention. Representative inflammatory mediators include agents which block, diminish, inhibit, or interfere with IL-1 activity, synthesis, or receptor signalling, such as anti-IL-1 antibody, soluble IL-LR, IL-1 receptor antagonist, or other appropriate peptides and small molecules; agents which block, diminish, inhibit, or interfere with IL-6 activity, synthesis, or receptor signalling, such as anti-IL-6 antibody, anti-gp 130, or other appropriate peptides and small molecules; modalities which block, diminish, inhibit, or interfere with the activity, synthesis, or receptor signalling of other inflammatory mediators, such as GM-CSF and members of the chemokine (IL-8) family; and cytokines with anti-inflammatory properties, such as IL-4, IL-10, and TGFβ. In addition, other anti-inflammatory agents, such as the anti-rheumatic agent methotrexate, can be administered in conjunction with the CD4+ T cell inhibiting agent and/or the TNF antagonist.

In one embodiment of the current invention, anti-CD4 antibody is used in conjunction with anti-TNF antibody. The term antibody is intended to encompass both polyclonal and monoclonal antibodies. The term antibody is also intended to encompass mixtures of more than one antibody reactive with CD4 or with TNF (e.g., a cocktail of different types of monoclonal antibodies reactive with CD4 or with TNF). The term antibody is further intended to encompass whole antibodies, biologically functional fragments thereof, bifunctional antibodies, and chimeric antibodies comprising portions from more than one species. Biologically functional antibody fragments which can be used are those fragments sufficient for binding of the antibody fragment to CD4 or to TNF.

The chimeric antibodies can comprise portions derived from two different species (e.g., human constant region and murine variable or binding region). The portions derived from two different species can be joined together chemically by conventional techniques or can be prepared as single contiguous proteins using genetic engineering techniques. DNA encoding the proteins of both the light chain and heavy chain portions of the chimeric antibody can be expressed as contiguous proteins.

Monoclonal antibodies reactive with CD4 or with TNF can be produced using somatic cell hybridization techniques (Kohler and Milstein, Nature 256: 495-497 (1975)) or other techniques. In a typical hybridization procedure, a crude or purified protein or peptide comprising at least a portion of CD4 or of TNF can be used as the immunogen. An animal is vaccinated with the immunogen to obtain anti-CD4 or anti-TNF antibody-producing spleen cells. The species of animal immunized will vary depending on the species of monoclonal antibody desired. The antibody producing cell is fused with an immortalizing cell (e.g., myeloma cell) to create a hybridoma capable of secreting anti-CD4 or anti-TNF antibodies. The unfused residual antibody-producing cells and immortalizing cells are eliminated. Hybridomas producing desired antibodies are selected using conventional techniques and the selected hybridomas are cloned and cultured.

Polyclonal antibodies can be prepared by immunizing an animal with a crude or purified protein or peptide comprising at least a portion of CD4 or of TNF. The animal is maintained under conditions whereby antibodies reactive with either CD4 or TNF are produced. Blood is collected from the animal upon reaching a desired titre of antibodies. The serum containing the polyclonal antibodies (antisera) is separated from the other blood components. The polyclonal antibody-containing serum can optionally be further separated into fractions of particular types of antibodies (e.g., IgG, IgM).

Antibodies specific for CD4 have been used in treatment of a wide range of both experitentally-induced and spontaneously-occurring autoimmune diseases. A more detailed description of anti-CD4 antibodies and their use in treatment of disease is contained in the following references, the teachings of which are hence incorporated by reference: U.S. application Ser. No. 07/867,100, filed Jun. 25, 1992; Grayheb, J. et al., J. of Autoimmunity 2:627-642 (1989); Ranges, G. E. et al, J. Exp. Med. 162: 1105-1110 (1985); Hom, J. T. et al., Eur. J. Immunol. 18: 881-888 (1988); Wooley, P. H. et al., J. Immunol. 134: 2366-2374 (1985); Cooper, S. M. et al., J. Immunol, 141: 1958-1962 (1988); Van den Broek, M. F. et al., Eur. J. Immunol. 22: 57-61 (1992); Wofsy, D. et al., J. Immunol.134: 852-857 (1985); Wofsy, D. et al., J. Immunol., 136: 4554-4560 (1986); Ermak, T. J. et al., Laboratory Investigation 61: 447-456 (1989); Reiter, C. et al., 34:525-532 (1991); Herzog, C. et al., J. Autoimmun. 2:627 (1989); Ouyang, Q. et al., Dig. Dis. Sci. 33:1528-1536 (1988); Herzog, C. et al., Lancet, p. 1461 (Dec. 19, 1987); Emmrich, J. et al., Lancet 338:570-571 (Aug. 31, 1991).

A more detailed description of anti-TNF antibodies and their use in treatment of disease is contained in the following references, the teachings of which are hence incorporated by reference; U.S. application Ser. No. 07/943,852, filed Sep. 11, 1992; Rubin et al., (EPO Patent Publication 0218868, Apr. 22, 1987); Yone et al., (EPO Patent Publication 0288088, Oct. 26, 1988); Liang, C.-M. et al., Biochem. Biophys. Res. Comm. 137:847-854 (1986); Meager, A. et al., Hybridoma 6:305-311 (1987); Fendly et al., Hybridoma 6:359-369 (1987); Bringman, T. S. et al., Hybridoma 6:489-507 (1927); Bringman T. S. et al., Hybridoma 6:489-507 (1987); Hirai, M. et al., J. Immunol. Meth. 96:57-62 (1987); Moller, A. et al., Cytokine 2:162-169 (1990); Mathison, J. C. et al., J. Clin. Invest. 81:1925-1937 (1988); Beutler, B. et al., Science 229:869-871 (1985); Tracey, K. J. et al., Nature 330:662-664 (1987); Shimamoto, Y. et al., Immunol. Lett. 17:311-318 (1988); Silva, A. T. et al., J. Infect. Dis. 162: 421-427 (1990); Opal, S. M. et al., J. Infect. Dis. 161:1148-1152 (1990); Hinshaw, L. B. et al., Circ. Shock 30:279-292 (1990); Lancet 342:173-174 (1993); Williams, R. O. et al., Proc. Natl. Acad. Sci. USA 89:9784-9788 (1992).

The CD4+ T cell inhibiting agent and TNF antagonist can be administered by various routes, including subcutaneously, intravenously, intramuscularly, topically, orally, rectally, nasally, buccally, vaginally, by inhalation spray, or via an implanted reservoir in dosage formulations containing conventional non-toxic pharmaceutically-acceptable carriers, adjuvants and vehicles. The form in which the agents are administered (e.g., capsule, tablet, solution, emulsion) will depend at least in part on the route by which it is administered.

A therapeutically effective amount of the combination of anti-CD4 agent and anti-TNF agent is that amount necessary to significantly reduce or eliminate symptoms associated with a particular autoimmune or inflammatory disorder. The therapeutically effective amount will be determined on an individual basis and will be based, at least in part, on consideration of particular agents used, the individual's size, the severity of symptoms to be treated, the result sought, etc. In one embodiment, for example, the preferred therapeutically effective amount of anti-CD4 antibody administered in conjunction with anti-TNF antibody is in the range of 0.1-10 mg/kg/dose of each antibody. Thus, the therapeutically effective amount can be determined by one of ordinary skill in the art employing such factors and using no more than routine experimentation.

The therapeutically effective amount can be administered in the form of a single dose, or a series of doses separated by intervals of days or weeks. Once the therapeutically effective amount has been administered, a maintenance amount of anti-CD4 agent, of anti-TNF agent, or of a combination of anti-CD4 agent and anti-TNF agent can be administered. A maintenance amount is the amount of anti-CD4 agent, anti-TNF agent, or combination of anti-CD4 agent and anti-TNF agent necessary to maintain the reduction or elimination of symptoms achieved by the therapeutically effective dose. The maintenance amount can be administered in the form of a single dose, or a series or doses separated by intervals of days or weeks. Like the therapeutically effective amount, the maintenance amount will be determined on an individual basis.

The combination therapy of the current invention is thus useful for the treatment of many autoimmune or inflammatory diseases of humans and of animals. In humans, diseases for which the therapy is appropriate include rheumatoid arthritis (RA) and juvenile chronic arthritis (JCA). Other diseases and conditions for which combination therapy is appropriate include spondyloarthropathies, such as ankylosing spondylitis, psoriatic arthritis, or arthritis associated with inflammatory bowel disease; vasculitides, such as polyarteritis nodosa, Wegener's granulomatosis, giant cell arteritis, Henoch Schoeniein purpura, and microscopic vasculitis of the kidneys; Sjogren's syndrome; systemic lupus erythemtatosus; inflammatory bowel disease, including Crohn's disease and ulcerative colitis; chronic active hepatitis; primary biliary cirrhosis; cryptogenic fibrosing alveolitis and other fibrotic lung diseases; uveitis; multiple sclerosis; myasthenia gravis; hemolytic anemia; scleroderma; graft versus host disease; allergy; and transplantation of kidneys, liver, heart, lungs, bone marrow, skin, or of other organs.

The invention is further and more specifically illustrated by the following Examples.

### EXAMPLE 1

### Treatment of Induced Arthritis in a Murine Model usina Anti-CD4 Antibody and Anti-TNF Antibody

The murine model of collagen type II induced arthritis has similarities to rheumatoid arthritis (RA) in its marked MHC class II predisposition, as well as in histology, immunohistology, and erosions of cartilage and bone. Furthermore, there is a good correlation of therapeutic response with human rheumatoid arthritis. For example, in both diseases anti-TNF antibody has beneficial effects (Williams, R. O. et al., PNAS 89:9784-9788 (1992); Elliott, M. J. et al., Arthritis & Rheumatism 36:1681-90 1993), and anti-CD4 antibody has minimal effect (Williams, R. O. et al., PNAS (in press) (1994); and Horneff, G. et al., Arthritis & Rheumatism 1991:34-129 (1992)). Thus the animal model serves as a good approximation to human disease.

The model of rheumatoid arthritis used herein is described by Williams, R. O. et al., (PNAS, 89:9784-9788 (1992), i.e., the collagen type II induced arthritis in the DBA/1 mouse. Type II collagen was purified from bovine articular cartilage by limited pepsin solubilization and salt fractionation as described by Miller (Biochemistry 11:4903-4909 (1972)).

A. Study 1

Male DBA/1 mice were immunized intradermally at 8-12 weeks of age with 100 μg of bovine type II collagen emulsified in complete Freund's adjuvant (Difco Laboratories, East Molsey, UK), and 21 days later with 100 μg of collagen intra-peritoneally (i.p.). Immediately after the onset of clinically evident arthritis (redness and/or swelling in one or more limbs), which was about 35 days after the initial injection, mice were injected i.p. with anti-CD4; anti-TNF; anti-CD4 and anti-TNF; or isotype controls. Arthritis was monitored for clinical score and paw-swelling for 10 days. Antibody treatment was administered on day 1 (onset), day 4 and day 7.

Clinical Score and Pawswelling

Two experiments were completed, assessing clinical score and pawswelling. In each, 200 μg of anti-CD4 were used per injection (rat YTS 191 and YTA 3.1) was used. Clinical score was assessed on the following scale: 0=normal; 1=slight swelling and/or erythema; 2 =pronounced edematoma swelling; and 3=joint rigidity. Each limb was graded, giving a maximum score of 12 per mouse. Pawswelling was monitored by measuring the thickness of each affected hind paw with calipers. The results were expressed as the percentage increment in paw width relative to the paw width before the onset of arthritis.

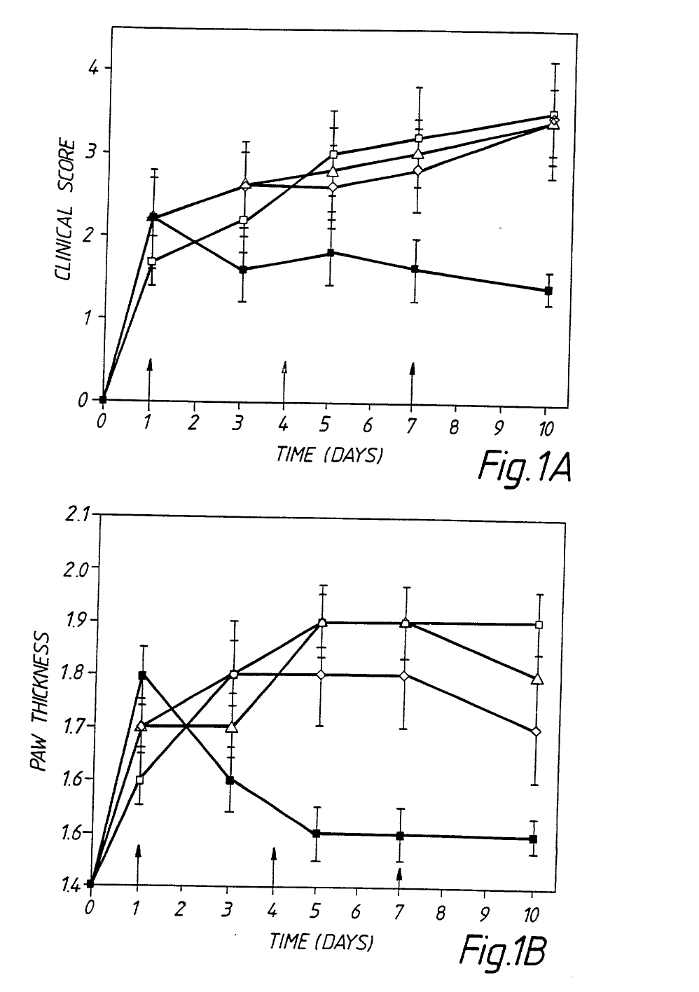

In the first experiment, a single dose of 50 μg per injection of anti-TNF (hamster TN3.19.2) was administered to each of five mice per group. There was no significant effect of anti-CD4 or anti-TNF (TN3.19 given 3 times at 50 μg/mouse). Hence the benefit of combination therapy, in both clinical score and footpad swelling, is readily seen (see FIGS. 1A, 1B).

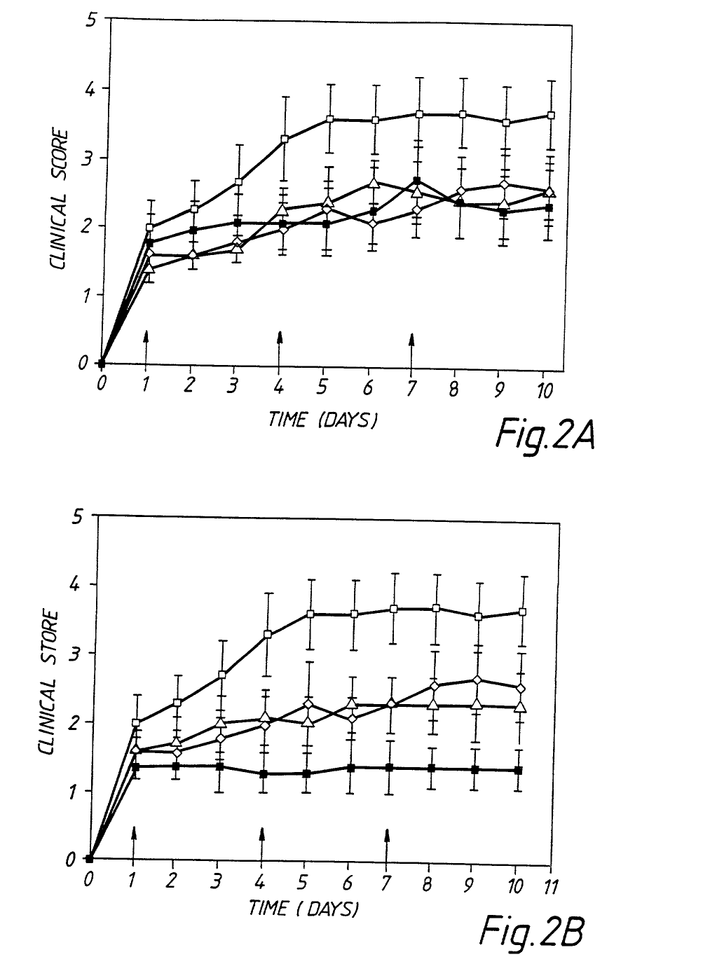

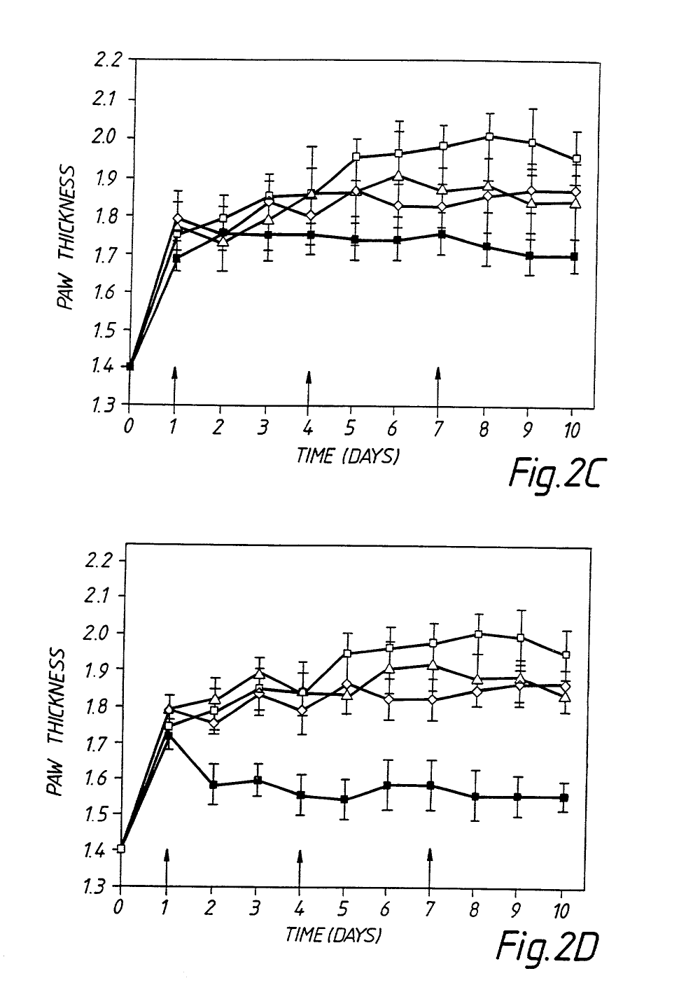

In the second experiment, either 50 μg or 300 μg of anti-TNF were administered to each of 7 mice per group. Both anti-CD4 and anti-TNF at low (50 μg) concentration had some effect, and benef it of combination therapy of these two concentrations was noted in pawswelling, not in clinical score. However, if anti-TNF was injected at 300 μg/mouse, the benefit of combination therapy with anti-CD4 was seen in both clinical score and more clearly in paw-swelling (see FIGS. 2A, 2B, 2C, 2D).

The results of the experiments indicate that there is a clear benefit to combination therapy with anti-TNF and anti-CD4 antibodies, as measured by clinical score and foot pad swelling.

B. Study 2

Male DBA/1 mice were immunized intradermally at 8-12 weeks of age with 100 μg type II collagen emulsified in Freund's complete adjuvant (Difco Laboratories, East Molsey, UK). Day one of arthritis was considered to be the day that erythema and/or swelling was first observed in one or more limbs. Arthritis became clinically evident around 30 days after immunization with type II collagen. For each mouse, treatment was started on the first day that arthritis was observed and continued over a 10 day period, after which the mice were sacrificed and joints were processed for histology. Monoclonal antibody (mAb) treatment was administered on days 1, 4, and 7. For anti-TNF antibody, TN3-19.12, a neutralizing hamster IgG1 anti-TNFα/β monoclonal antibody (mAb), was used (Sheehan, K. C. et al., J. Immunology 142:3884-3893 (1989)). The isotype control was L2. The anti-TNF antibody and the isotype control were provided by R. Schreiber, Washington University Medical School (St. Louis, Mo., USA), in conjunction with Celltech (slough, UK). The cell-depleting anti-CD4 monoclonal antibody (rat IgG2b) consisted of a 1:1 mixture of YTS 191.1.2 and YTA 3.1.2, provided by H. Waldmann (University of Cambridge, UK) (Galfre, G. et al., Nature 277: 131-133 (1979); Cobbold, S. P. et al., Nature 31z: 548-551 (1984); Qin, S. et al., European J. Immunology 17:1159-1165 (1987)).

Paw-Swelling

First, a sub-optimal dose of 50 μg of anti-TNF alone was compared with the same dose given together with 200 μg of anti-CD4. To verify the results, two separate but identical experiments were carried out (11-12 mice/group and 7-8 mice/group, respectively). Neither anti-CD4 alone nor sub-optimal anti-TNF alone were able to significantly reduce paw-swelling (data not shown). However, treatment with anti-TNF and anti-CD4 resulted in a consistently and statistically significant reduction in paw-swelling relative to the group given control mAb (P<0.001). Furthermore, in both experiments, combined anti-TNF/anti-CD4 treatment (also referred to herein as anti-CD4/TNF treatment) produced a significant reduction in paw-swelling relative to anti-CD4 alone, and anti-TNF alone (P<0.05).

Next, an optimal dose of anti-TNF (300 μg) alone was compared in two separate but identical experiments (7-7 mice/group and 6-7 mice/group, respectively) with the same dose given in combination with anti-CD4. As before, the combined anti-TNF/anti-CD4 treatment resulted in a significant reduction in paw-swelling compared to treatment with the control mDb (P<0.005; data not shown). In the first experiment, paw-swelling was also significantly reduced in the combined anti-CD4/anti-TNF treated group relative to the groups given anti-CD4 alone or anti-TNF alone (P<0.05). Some reduction in paw-swelling was observed in mice given either anti-TNF alone or anti-CD4 alone although the differences were not significant, possibly because of the small group sizes (6 per group). In the second experiment, combined anti-CD4/anti-TNF gave significantly reduced paw-swelling compared to anti-CD4 alone (P<0.05) but not compared to anti-TNF alone since anti-TNF itself caused a significant reduction in paw-swelling, as expected from previous work (Williams, R. O. et al., PNAS 89: 9784-9788 (1992)). In the experiments, the reduction in paw-swelling attributable to anti-TNF alone was 23% and 33%, respectively. Thus, the reduction in paw-swelling attributable to anti-TNF treatment was broadly comparable with our previously published findings in which treatment with TN3-119.12 (300 μg/mouse) resulted in a mean reduction in paw-swelling over the treatment period of around 34% relative to controls (Williams, R. O. et al., PNAS 89: 9784-9788 (1992)).

Limb Involvement

In collagen-induced arthritis, as in RA, it is usual for additional limbs to become involved after the initial appearance of clinical disease and new limb involvement is an important indicator of the progression of the disease. To determine the effect of anti-CD4/anti-TNF treatment on new limb involvement, the number of limbs with clinically detectable arthritis at the end of the 10 day treatment period was compared with the number of arthritis limbs before treatment. In mmice given the control mAb there was an increase in limb involvement over the 10 day period of approximately 50% The results from the two experiments were pooled, and are shown in Table 1.

TABLE 1
Combined anti-CD4/anti-TNF Inhibits Progression
of Clinical Arthritis

| | Number of Limbs Affected | | |
|---|---|---|---|
| | (Mean ± SEM) | | Increase |
| Treatment | Day 1 | Day 10 | (%) |

Sub-optimal anti-TNF (50 μg)

| | | | |
|---|---|---|---|
| anti-CD4 | 1.30 ± 0.10 | 1.90 ± 0.13 | 46.1 |
| (n = 18) | | | |
| anti-TNF | 1.20 ± 0.09 | 1.65 ± 0.17 | 37.5 |
| (n = 19) | | | |
| anti-CD4/TNF | 1.40 ± 0.09 | 1.45 ± 0.22 | 3.4¹ |
| (n = 18) | | | |
| control mAb | 1.43 ± 0.15 | 2.24 ± 0.18 | 56.6 |
| (n = 18) | | | |

Optimal anti-TNF (300 μg)

| | | | |
|---|---|---|---|
| anti-CD4 | 1.27 ± 0.10 | 1.80 ± 0.14 | 42.0 |
| (n = 12) | | | |
| anti-TNF | 1.50 ± 0.17 | 1.64 ± 0.20 | 9.5² |
| (n = 11) | | | |
| anti-CD4/TNF | 1.25 ± 0.11 | 1.25 ± 0.11 | 0³ |
| (n = 13) | | | |
| control mAb | 1.53 ± 0.19 | 2.27 ± 0.25 | 47.8 |
| (n = 12) | | | |

¹P < 0.05 (anti-CD4/TNF vs. control mAb)
²P < 0.05 (anti-TNF vs. control mAb)
³P < 0.005 (anti-CD4/TNF vs. control mAb)

There was some reduction in new limb involvement in the groups given anti-CD4 alone and sub-optimal anti-TNF alone, although the differences were not significant. In the group given optimal anti-TNF the increase in limb involvement was less than 10% (P<0.05). More striking, however, was the almost complete absence of new limb involvement in the groups given combined anti-CD4/anti-TNF. Thus, the increase in new limb involvement was only 3% in mice given anti-CD4 plus suboptimal anti-TNF (P<0.05) and 0% in mice given anti-CD4 plus optimal anti-THP (P<0.005).

Histology

After 10 days, the mice were sacrificed; the first limb that had shown clinical evidence of arthritis was removed from each mouse, formalin-fixed, decalcified, and wax-embedded before sectioning and staining with haematoxylon and eosin. A sagittal section of the proximal interphalangeal (PIP) joint of the middle digit was studied in a blind fashion for the presence or absence of erosions in either cartilage or bone (defined as demarcated defects in cartilage or bone filled with inflammatory tissue). The comparisons were made only between the same joints, and the arthritis was of identical duration. Erosions were observed in almost 100% of the PIP joints from the control groups and in approximately 70-80% of the joints given either anti-CD4 alone or sub-optimal anti-TNF alone. The results of the two experiments were pooled, and are shown in Table 2.

TABLE 2
Proportions ot PIP Joints Showing
Significant Erosion of Cartilage and/or
Bone

| | |
|---|---|
| | Joints with |
| Treatment | Erosions |

Sub-optimal anti-TNF (50 μg)

| | | |
|---|---|---|
| anti-CD4 | 13/18 | (72%) |
| anti-TNF | 14/19 | (74%) |
| anti-CD4/TNF | 4/18 | (22%)¹ |
| control mAb | 17/18 | (94%) |

Optimal anti-TNF (300 μg)

| | | |
|---|---|---|
| anti-CD4 | 10/12 | (83%) |
| anti-TNF | 6/11 | (54%)² |
| anti-CD4/TNF | 4/13 | (31%)³ |
| control mAb | 12/12 | (100%) |

¹P < 0.01 (anti-CD4/TNF vs. anti-CD4 alone; anti-TNF alone and control mAb)
²P < 0.01 (anti-TNF alone vs. control mAb)
³P < 0.01 (anti-CD4/TNF vs. anti-CD4 alone and control mAb)

An optimal dose of anti-TNF alone significantly reduced pathology, as reported previously (Williams, R. O. et al., PNAS 89: 9784-9788 (1992)). Thus, in the mice given optimal anti-TNF alone the proportion of joints showing. erosive changes was reduced to 54% (P<0.001) whereas in the groups given anti-CD4 plus either sub-optimal or optimal anti-TNF,only 22% (P<0.01) and 31% (P>0.01) of the joints, respectively, were eroded. Thus, 300 μg of anti-TNF alone gave a degree of protection against joint erosion but combined anti-CD4/anti-TNF provided significantly greater protection.

Depletion ot CD4+ T Cells

The extent to which anti-CD4 treatment depleted peripheral CD4+ T cells was determined by flow cytometry. To enumerate the proportion of CD4+lymphocytes in disassociated spleen populations or peripheral blood, cells were incubated with phycoerythrin-conjugated anti-CD4 (Becton Dickinson, Oxford, UK), then analyzed by flow cytometry. (FACSean, Becton Dickinson) with scatter gates set on the lymphocyte fraction. Anti-CD4 treatment resulted in 98% (±1%) depletion of CD4+ T cells in the spleen and 96% (±3%) depletion of CD4+ T cells in the blood.

Immunohistochemistry

The possible persistence of CD4+ T cells in the joint despite virtual elimination of peripheral CD4+ T cells was next investigated by immunohistochemical analysis of sections from treated arthritic mice. Wax-embedded sections were de-waxed, trypsin digested, then incubated with anti-CD4 mAb (YTS 191.1.2/YTA 3.1.2). To confirm the T cell identity of the CD4+ T cells, sequential sections were stained with anti-Thy-1 mAb (YTS 154.7) (Cobbold, S. P. et al., Nature 312:548 -551 (1984)). Control sections were incubated with HRPN11/12a. Detection of bound antibody was by alkaline phosphatase/rat anti-alkaline phosphatase complex (APAA; Dako, High Wycombe, UK) and fast red substrate as described (Deleuran, B. W. at al., Arthritis & Rheumatism 34:1125-1132 (1991)). Small numbers of CD4+ T cells were detected in the joints, not only of mice given control mAb, but also of those treated with anti-CD4 (data not shown). Furthermore, within the small number of nice that were studied (four per treatment group), it was not possible to detect significantly reduced numbers of CD4+ T cells in the groups given anti-CD4 alone or anti-CD4 plus anti-TNF (data not shown). Anti-CD4 treatment did not, therefore, eliminate CD4+ T cells from the joint.

Anti-collagen IgG Levels

Serum anti-collagen IgG levels were measured by enzyme-linked immunosorbent assay (ELISA). Microtitre plates were coated with bovine type II collagen (2 μg/ml), blocked, then incubated with test sera in serial dilution steps. Detection of bound IgG was by incubation with alkaline phosphatase-conjugated goat anti-mouse IgG, followed by substrate (dinitrophenol phosphate). Optical densities were read at 405 nm. A reference sample, consisting of affinity-purified mouse anti-type II collagen antibody, was included on each plate. Results are shown in Table 3.

TABLE 3
Serum Levels of Anti-type II collagen IgG

| | |
|---|---|
| | Anti-collagen IgG |
| Treatment | (Mean ± SEM) (μg/ml) |

Sub-optimal anti-TNF (50 μg)

| | | |
|---|---|---|
| anti-CD4 | (n = 18) | 285 ± 37 |
| anti-TNF | (n = 19) | 208 ± 29 |
| anti-CD4/TNF | (n = 18) | 208 ± 34 |
| control mAb | (n = 18) | 238 ± 36 |

Optimal anti-TNF (300 μg)

| | | |
|---|---|---|
| anti-CD4 | (n = 12) | 288 ± 39 |
| anti-TNF | (n = 11) | 315 ± 49 |
| anti-CD4/TNF | (n = 12) | 203 ± 33 |
| control mAb | (n = 12) | 262 ± 47 |

Serum levels of anti-type II collagen IgG were not significantly altered within the 10 day treatment period by anti-CD4 alone, anti-TNF alone, or anti-CD4 plus anti-TNF.

Anti-Globulin Response

To find out whether anti-CD4 treatment prevented a neutralizing anti-globulin response against the anti-TNF mAb, IgM anti-TN4-19.12 levels on day 10, as measured by ELISA, were compared. At this time, an IgG anti-TN3-19.12 response was not detected. Microtitre plates were coated with TN3-19.12 (5 μg/ml), blocked, then incubated with serially diluted test sera. Bound IgM was detected by goat anti-mouse IgM-alkaline phosphatase conjugate, followed by substrate. The results demonstrated that nti-CD4 was highly effective in preventing the evelopment of an anti-TN3-19.12 antibody response (Table 4). Next, to determine whether anti-CD4 treatment led to increased levels of circulating anti-TNF-α (by reducing the antibody response to the hamster anti-TNF), an ELISA was carried out in which recombinant murine TNF-α was used to detect free TN3-19.12 in the sera of mice on day 10 of the experiment. Microtitre plates ware coated with recombinant zurine TNF-α, blocked, then incubated with test sera. Coat anti-hamster IgG-alkaline phosphatase conjugate (adsorbed against murine IgG) was then applied, followed by substrate. Quantitation was by reference to a sample of known concentration of TN3-19.12. Results are shown in Table 4.

TABLE 4
IgM anti-TN3 Titres and Levels of Unbound TN3

| | | |
|---|---|---|
| | Reciprocal | Unbound TN3 |
| | of Anti-TN3 | (Mean ± SEM) |
| Treatment | Titre (Mean) | (μg/ml) |

Sub-optimal anti-TNF (50 μg)

| | | | |
|---|---|---|---|
| anti-TNF | (n = 12) | 242 | 8.6 ± 2.0 |
| anti-CD4/TNF | (n = 12) | 84¹ | 12.1 ± 1.9 |

Optimal anti-TNF (300 μg)

| | | | |
|---|---|---|---|
| anti-TNF | (n = 12) | 528 | 90.7 ± 11.9 |
| anti-CD4/TNF | (n = 12 | 91¹ | 102.7 ± 12.5 |

¹Significantly reduced anti-TN3 titre (P < 0.005)

Levels of TN3-19.12 were slightly elevated in the groups given anti-CD4 plus anti-TNF compared to anti-TNF alone, although the differences were not significantly different.

### EXAIPLE 2

### Treatment of Induced Arthritis in a Murine Model Using TNF Recegtor/IG Egusion Protein with Anti-CD4 Antibody

The murine model of collagen type II induced arthritis, described above, was used to investigate the efficacy of a human p55 TNF receptor/IgG fusion protein, in conjunction with anti-CD4 monoclonal antibody (mAb), for its ability to modulate the severity of joint disease in collagen-induced arthritis. First, a comparison was made between the efficacy of TNF receptor/IgG fusion protein treatment, anti-TNF mAb treatment, and high dose corticosteroid therapy. Subsequently, therapy with TNF receptor/IgG fusion protein in conjunction with anti-CD4 antibody was investigated.

A. Experimental Procedure

Male DBA/1 mice were immunized intradermally with 100 μg of bovine type II collagen emulsified in complete Freund's adjuvant (Difco Laboratories, East Molsey, UK). The mean day of onset of arthritis was approximately one month after immunization. After the onset of clinically evident arthritis (erythema and/or swelling), mice were injected intraperitoneally with therapeutic agents. Arthritis was monitored for clinical score and paw swelling (measured with calipers) for 10 days, after which the mice were sacrificed and joints were processed for histology. Sera were collected for analysis on day 10. Therapeutic agents were administered on day 1 (onset), day 4 and day 7. The therapeutic agents included TNF receptor/IgG fusion protein (p55-sf2), anti-TNF antibody, anti-CD4 antibody, and methylprednisolone acetate.

B. Comparison of Treatment with TNF Receptor/IgG Fusion Protein, Anti-TNF Antibody, or Methylprednisolone Acetate

Using the Experimental Procedure described above, groups of mice were subjected to treatment with TNF receptor/IgG protein (2 μg) (18 mice), TNF receptor/IgG protein (20 μg) (18 mice), TNF receptor/IgG protein (100 μg) (12 mice), anti-TNF monoclonal antibody (mAb) (300 μg) (17 mice), methylprednisolone acetate (6 mice), an irrelevant human IgG1 monoclonal antibody (mAb) (6 mice), or saline (control). The TNF receptor/IgG fusion protein, herein referred to as p55-sf2, (Butler et al., Cytokine (in press): (1994), was provided by Centocor, Inc., Malvern Pa.; it is dimeric and consists of the human p55 TNF receptor (extracellular domains) fused to a partial J sequence followed by the whole of the constant region of the hunan IgG1 heavy chain, itself associated with the constant region of a kappa light chain. The anti-TNP antibody was TN3-19.12, a neutralizing hamster IgG1 anti-TNFα/β monoclonal antibody (Sheehan, K. C. et al., J. Immunology 142:3884-3893 (1989)), and was provided by R. Schreiber, Washington University Medical School (St. Louis, Mo., USA), in conjunction with Celltech (Slough, UK). Neutralizing titres were defined as the concentration of TNFα neutralizing agent required to cause 50% inhibition of killing of WEHI 164 cells by trimeric recombinant murine TNFα; the neutralizing titre of p55-sf2 was 0.6 ng/ml, compared with 62.0 ng/ml for anti-TNF mAb (TN3-19.12), using 60 pg/ml mouse TNFα. The corticosteroid, mathyl-prednisolone acetate (Upjohn, Crawley, UK) was administered by intraperitoneal injection as an aqueous suspension at a dosage level of 2 mg/kg body weight; using the protocol described above, this dosage is equivalent to 4.2 mg/kg/week, a dose which is higher than the typical dose used to treat refractory RA in humans (1-2 mg/kg/week).

Paw-Swelling

Treatment with p55-sf2 resulted in a dose-dependent reduction in paw-swelling over the treatment period, with the doses of 20 μg and 100 μg giving statistically significant reductions in paw-swelling relative to mice given saline (P<0. 05). The group of mice given an irrelevant human IgG1 mAb as a control did not show any deviation from the saline-treated group (data not shown), indicating that the therapeutic effects of p55-sf2 were attributable to the TNF receptor rather than the human IgG1 constant region. Similar reductions in paw-swelling were seen in mice given 300 μg of anti-TNF mAb as in those given 100 μg of p55-sf2, although anti-TNF mAb was marginally more effective than p55-sf2 at inhibiting paw-swelling. A reduction in paw-swelling was observed in the methylprednisolone acetate treated group that was comparable in magnitude to the reductions given p55-sf2 at 100 μg or anti-TNF mAb at 300 μg.

Limb Involvement

The change in the number of arthritic limbs over the 10 day treatment period was examined. Results are shown in Table 5.

TABLE 5
Inhibitory Effect of TNF-Targeted Therapy on
Limb Recruitment

| | | Limbs Affected | | |
|---|---|---|---|---|
| Treatment (number | | (mean ± SEM) | | Increase |
| of animals) | | Day 1 | Day 10 | (%) |
| saline | (n = 12) | 1.33 ± 0.14 | 2.25 ± 0.18 | 69% |

| | | | |
|---|---|---|---|
| p55-sf2, | 1.28 ± 0.11 | 1.94 ± 0.17 | 51% |

| | | |
|---|---|---|
| 2 | μg | (n = 18) |

| | | | |
|---|---|---|---|
| p55-sf2, | 1.37 ± 0.11 | 1.79 ± 0.16 | 31% |

| | | |
|---|---|---|
| 20 | μg | (n = 18) |

| | | | |
|---|---|---|---|
| p55-sf2, | 1.17 ± 0.17 | 1.58 ± 0.23 | 35% |

| | | |
|---|---|---|
| 100 | μg | (n = 12) |

| | | | |
|---|---|---|---|
| Control IgG1, | 1.00 ± 0.00 | 0.15 ± 0.22 | 50% |

| | | |
|---|---|---|
| 100 | μg | (n = 6) |

| | | | |
|---|---|---|---|
| Anti-TNF mAb, | 1.47 ± 0.15 | 1.76 ± 0.16¹ | 20% |

| | | |
|---|---|---|
| 300 | μg | (n = 17) |

| | | | |
|---|---|---|---|
| Methylprednisolone | 1.00 ± 0.00 | 1.50 ± 0.22 | 33% |

| | |
|---|---|
| acetate | (n = 6) |

¹P < 0.05 (vs. saline; Mann Whitney Test)

A strong trend towards reduced limb recruitment was seen in the qroups of mice given p55-sf2, anti-TNF mAb or methylprednisolone acetate, but only in the anti-TNF mAb treated group did the reduction reach statistical sigrnificance (P<0.05).

Histology

After 10 days, the mice were sacrificed; the first limb to show clinical evidence of arthritis was removed from each mouse, fixed, decalcified, wax-embedded, and sectioned and stained with haematoxylon and eosin. Sagittal sections of the proximal interphalangeal (PIP) joint of the middle digit of each mouse were studied in a blind fashion and classified according to the presence or absence of erosions, as defined above. Comparisons were thus made between identical joints, and the arthritis was of equal duration. Results are shown in Table 6.

TABLE 6
Histopathology of PIP Joints

| Treatment | | | PIP Joints with Erosions | |
|---|---|---|---|---|
| Saline | | | 11/12 | (92%) |
| p55-sf2, | 2 | μg | 14/18 | (78%) |
| p55-sf2, | 20 | μg | 14/18 | (78%) |
| p55-sf2, | 100 | μg | 6/12 | (50%)¹ |
| Control IgG1, | 100 | μg | 6/6 | (100%) |
| Anti-TNF mAb, | 300 | μg | 7/17 | (41%)² |

| | | |
|---|---|---|
| Methylprednisolone acetate | 4/6 | (67%) |

¹P < 0.05 (vs. saline).
²P < 0.01 (vs. saline). Data were compared by Chi-square analysis.

Erosions were present in 92% and 100% of the PIP joints in the saline treated group and the control human IgG1 treated group, respectively. However, only 50% (P<0.05) of joints from the mice treated with p55-sf2 (100 μg) and 41% (P<0.01) of mice given anti-TNF mAb exhibited erosive changes. Some reductions in the proportion of eroded joints were observed in mice treated with 2 μg or 20 μg of p55-sf2, but these were not statistically significant. Similarly, treatment with methylprednisolone acetate did not significantly reduce joint erosion.

Anti-Collagen Antibody Levels

Anti-collagen IgG levels on day 10 were measured by ELISA as described (Williams, R. O. et al., PNAS 89: 9784-9788 (1992)). Microtitre plates were sensitized with type II collagen, then incubated with serially-diluted test sera. Bound IgG was detected using alkaline phosphatase-conjugated goat anti-mouse IgG, followed by substrate (dinitrophenol phosphate). Optical densities were read at 405 nm. No differences between any of the treatment groups were detected (data not shown). This suggests that the therapeutic effect of p55-sf2 is not due to a generalized immunosuppressive effect.

C. Effect of Treatment with P55-sf2 in Conjunction with Anti-CDL4 Antibody

In view of the high titres of antibodies to p55-sf2 that were detected in mice treated with the fusion protein, an experiment was carried out to determine whether concurrent administration of anti-CD4 monoclonal antibody (mAb) could enhance the therapeutic effects of p55-sf2. Using the Experimental Procedure described above, a comparison was made of three different treatment regimes: anti-CD4 mAb alone (200 μg), p55-sf2 alone (100 μg) or anti-CD4 mAb (200 μg) plus p55-sf2 (100 μg). A fourth group consisted of untreated control mice. The cell-depleting anti-CD4 b (rat IgG2b) consisted of a 1:1 mixture of YTS 191.1.2 and YTA 3.1.2, provided by H. Waldmann (University of Cambridge, UK) (Galfre, G. et al., Nature 277: 131-133 (1979); Cobbold, S. P. et al., Nature 312: 548-551 (1984); Qin, S. et al., European J. Immunology 17:1159-1165 (1987)). p55-sf2 is described above.

Paw-Swelling

Treatment with p55-sf2 alone resulted in a marked inhibition of paw-swelling, but the synergistic inhibitory effect of anti-CD4 mAb in combination with p55-sf2 was remarkable. In contrast, anti-CD4 mib treatment alone had very little effect on paw-swelling.

Limb Involvement

As before, the progressive involvement of additional limbs following the initial appearance of arthritis was studied. Results are shown in Table 7.

TABLE 7
Anti-CD4 Antibody and p55-sf2 Prevent New Limb
Recruitment

| | | Limbs Affected | | |
|---|---|---|---|---|
| Treatment (number | | (mean ± SEM) | | Increase |
| of animals) | | Day 1 | Day 10 | (%) |
| Control | (n = 6) | 1.17 ± 0.17 | 2.00 ± 0.26 | 71% |
| Anti-CD4 mAb | (n = 6) | 1.17 ± 0.17 | 1.83 ± 0.31 | 56% |
| p55-sf2 | (n = 7) | 1.43 ± 0.20 | 1.71 ± 0.18 | 19% |

| | | | |
|---|---|---|---|
| Anti-CD4 mAb/ | 1.33 ± 0.21 | 1.33 ± 0.21¹ | 0% |

| | |
|---|---|
| p55-sf2 | (n = 6) |

¹P < 0.05 (vs. controls; Mann whitney test).

There was a mean increase in limb involvement of 71% in the control group, which was reduced to 56% in the group given anti-CD4 b alone, and only 19% in the group given p55-sf2. However, in the group given anti-CD4 mAb plus p55-sf2, the increase in limb involvement was 0%, a statistically significant difference.

Histology

Histological analysis of PIP joints of treated mice was carried out as described above. Results are shown in Table 8.

TABLE 8
Effects of Anti-CD4 mAb arid p55-sf2 in the
Prevention of Joint Erosion

| Treatment | PIP Joints with Erosions | |
|---|---|---|
| control | 6/6 | (100%) |
| Anti-CD4 mAb | 6/6 | (100%) |
| p55-sf2 | 2/6 | (33%)¹ |
| Anti-CD4 mAb plus p55-sf2 | 1/6 | (17%)² |

¹P = 0.06 (vs. control)
²P < 0.05 (vs. control) Data were compared by the Fisher exact test.

The control group and the group given anti-CD4 mob alone gave identical results, with 6/6 (100%) of PIP joints in both groups showing significant erosions. However, in the group given p55-sf2 alone, only 2/6 (33%) of PIP joints showed erosions. Only ⅙ (17%) of joints showed erosions in the group given anti-CD4 plus p55-sf2.

Antibody Responses to p55-sf2

The IgM/IgG responses to injected p55-sf2 were measured by ELISA at the end of the treatment period (day 10). Microtitre plates were coated with p55-sf2 (5 μg/ml), blocked, then incubated with serially diluted test sera. Negative controls consisted of sera from saline-treated mice. Bound IgM or IgG were detected by the appropriate goat anti-mouse Ig-alkaline phosphatase conjugate, followed by substrate. Results are shown in Table 9.

TABLE 9
Anti-p55-sf2 Responses and Levels of Free p55-
sf2 in Sera of Mice Treated with p55-sf2 Alone
or in combination with Anti-CD4 mAb

| | Anti-p55-sf2 | | |
|---|---|---|---|
| | Response (titres) | | |
| Treatment | IgM | IgG | p55-sf2 Level |

Experiment 1

| saline | 1:20 | 1:35 | — | |
|---|---|---|---|---|
| p55-sf2, | 1:50 | 1:590 | <0.2 | μg/ml |

| | |
|---|---|
| 2 | μg |

| | | | | |
|---|---|---|---|---|
| p55-sf2, | 1:232 | 1:3924 | <0.2 | μg/ml |

| | |
|---|---|
| 20 | μg |

| | | | | |
|---|---|---|---|---|
| p55-sf2, | 1:256 | 1:5280 | <0.2 | μg/ml |

| | |
|---|---|
| 100 | μg |

Experiment 2

| | | | | |
|---|---|---|---|---|
| p55-sf2, | 1:336 | 1:5100 | <0.2 | μg/ml |

| | |
|---|---|
| 100 | μg |

| | | | | |
|---|---|---|---|---|
| p55-sf2, | 1:15 | 1:200 | 12.3 ± 1.1 | μg/ml |

| | |
|---|---|
| 100 | μg, |

plus
anti-CD4 mAb

High titres of both IgM and IgG antibodies to p55-sf2 were detected in treated mice, with the highest titres being found in the mice given the 100 μg dose. These results indicate that p55-sf2, which is derived from human proteins, is highly immunogenic in mice. This may account for the slightly greater efficacy of anti-TNF mAb in vivo described in Section B, above, despite the higher neutralizing titre of the fusion protein in vitro. Anti-CD4 mAb treatment was found to block almost completely the formation of both IgM and IgG antibodies to p55-sf2.

Serum Levels of Free p55-sf2

Microtitre plates were coated with recombinant murine TNF-α (Genentech Inc., San Francisco, Calif.), blocked, then incubated with test sera. Goat anti-human IgG-alkaline phosphatase conjugate was then applied followed by substrate. Quantitation was by reference to a sample of known concentration of p55-sf2.

The inhibition of the antibody response was associated with pronounced differences in the circulating levels of p55-sf2 in treated mice. Thus, free p55-sf2 was undetectable in mice given the fusion protein alone, whereas in the mice given anti-CD4 mAb plus p55-sf2, the mean serum level of p55-sf2 was 12.3 μg/ml.

### EXAMPLE3

### Treatment of Induced Arthritis in a Murine Model Using Cyglosporin A and Anti-TNF Antibody

The murine model of collagen type II induced arthritis, described above, was used to investigate the efficacy of the CD4+ T cell inhibiting agent cyclosporin A in conjunction with anti-TNF monoclonal antibody (mAb), for the ability to modulate the severity of joint disease in collagen-induced arthritis. A comparison was made between the efficacy of treatment with cyclosporin A (CsA), anti-TIF antibody, and combination of CSA and anti-TNF antibody.

A. Experimental Procedure

Male DBA/1 mice were immunized intradermally with 100 μg of bovine type II collagen emulsified in complete Freund's adjuvant (Difco Laboratories, East Molsey, UK). The mean day of onset of arthritis was approximately one month after immunization. After the onset of clinically evident arthritis (erythema and/or swelling), groups of mice (11 mice each) were subjected to treatment with one of the following therapies: 50 μg (2 ag/kg) L2 (the isotype control for anti-TNF antibody), intraperitoneally once every three days (days 1, 4 and 7); 250 μg (10 mg/kg) cyclosporin A intraperitoneally daily; 50 μg (2 mg/kg) anti-TNF mAb TN3-19.12, intraperitoneally once every three days (days 1, 4 and 7); or 250 μg cyclosporin A intraperitoneally daily in conjunction with 50 μg anti-TNF mAb intraperitoneally once every three days. Arthritis was monitored for paw swelling (measured with calipers) for 10 days, after which the mice were sacrificed and joints were processed for histology.

Paw-Swelling

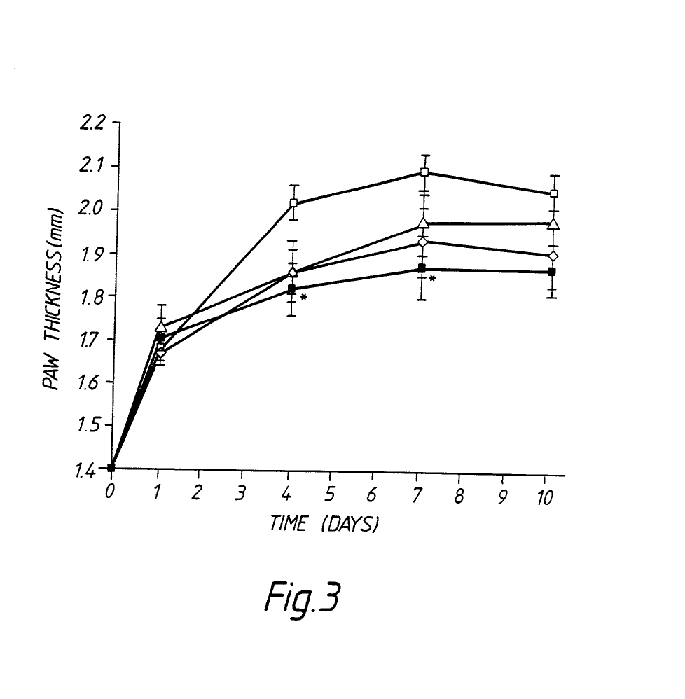

Treatment with cyclosporin A in conjunction with anti-TNF mAb resulted in a reduction in paw-swelling over the treatment period, relative to mice treated with control antibody. Results are shown in FIG. 3.

Limb Involvement

As before, the progressive involvement of additional limbs following the initial appearance of arthritis was studied. Results are shown in Table 10.

TABLE 10
Anti-CD4 Antibody and p55-sf2 Prevent New Limb
Recruitrrient

| | Limbs Affected | | |
|---|---|---|---|
| | (mean ± SEM) | | Increase |
| Treatment | Day 1 | Day 10 | (%) |
| Control mAb | 1.36 ± 0.20 | 2.45 ± 0.28 | 80.1% |
| Cyclosporin A | 1.36 ± 0.15 | 2.18 ± 0.30 | 60.3% |
| Anti-TNF mAb | 1.45 ± 0.16 | 1.9 ± 0.21 | 31.0% |
| CsA/Anti-TNf mAb | 1.27 ± 0.14 | 1.54 ± 0.20¹ | 21.0% |

¹P = 0.03 vs. control).

Treatment with cyclosporin A in conjunction with anti-TNF mAb resulted in statistically significant reductions in limb involvement in comparison to control monoclonal antibody (P=0.03).

Equivalents

Those skilled in the art will recognize, or be able to ascertain using no more than routine experimentation, many equivalents to specific embodiments of the invention described specifically herein. Such equivalents are intended to be encompassed in the scope of the following claims.

What is claimed is:

1. A method of treating autoimmune or inflammatory disease in a mammal comprising administering to said mammal a therapeutically effective amount of a combination of a CD4+ T cell inhibiting agent and a TNF antagonist.

2. A method of claim 1, wherein the CD4+ T cell inhibiting agent is administered simultaneously with the TNF antagonist.

3. A method of claim 1, wherein the CD4+ T cell inhibiting agent is administered sequentially with the TNF antagonist.

4. A method of claim 1, wherein the CD4+ T cell inhibiting agnet and the TNF antagonist are administered by a route selected from the group consisting of: subcutaneously, intravenously, and intramuscularly.

5. A method of claim 1, wherein the CD4+ T cell inhibiting agent and the TNF antagonist are administered in a pharmaceutically acceptable vehicle.

6. A method of claim 1, wherein an anti-inflammatory agent is administered in conjunction with the CD4+ T cell inhibiting agent and the TNF antagonist.

7. A method of claim 6, wherein the anti-inflammatory agent is an agent interfering with the activity or synthesis of TNF.

8. A method of claim 6, wherein the anti-inflammatory agent is an agent interfering with the activity or synthesis of IL-1.

9. A method of claim 6, wherein the anti-inflammatory agent is an agent interfering with the activity or synthesis of IL-6.

10. A method of claim 6, wherein the anti-inflammatory agent is a cytokine with anti-inflammatory properties.

11. A method of claim 1, wherein the autoimmune disease is rheumatoid arthritis.

12. A method of claim 1, wherein the CD4+ T cell inhibiting agent is an antibody to T cells or to T cell receptors.

13. A method of claim 1, wherein the CD4+ T cell inhibiting agent is an antibody to an antigen presenting cell or to the receptors of an antibody presenting cell.

14. A method of claim 1, wherein the CD4+ T cell inhibiting agent is a peptide or small molecule which inhibits T cell interaction with antigen presenting cells.

15. A method of treating autoimmune or inflammatory disease in a mammal comprising administering to said mammal a therapeutically effective amount of a combination of a CD4+ T cell inhibiting agent and an inflammatory mediator which down-regulates cytokines.

16. A method of claim 15, wherein the inflammatory mediator is agent interfering with the activity or synthesis of TNF.

17. A method of claim 15, wherein the inflammatory mediator is an agent interfering with the activity or synthesis of IL-1.

18. A method of claim 15, wherein the inflammatory mediator is an agent interfering with the activity or synthesis of IL-6.

19. A method of claim 15, wherein the inflammatory mediator is a cytokine with anti-inflammatory properties.

20. A method of treating autoimmune or inflammatory disease in a mammal, comprising administering to said mammal a therapeutically effective amount of a combination of anti-CD4 antibody and anti-TNF antibody.

21. A method of treating autoimmune or inflammatory disease in a mammal, comprising administering to said mammal a therapeutically effective amount of a combination of anti-CD4 antibody and soluble TNF receptor.

22. A method of treating autoimmune or inflammatory disease in a mammal, comprising administering to said mammal a therapeutically effective amount of a combination of anti-CD4 antibody and TNF receptor/IgG fusion protein.

23. A method of treating autoimmune or inflammatory disease in a mammal, comprising administering to said mammal a therapeutically effective amount of a combination of cyclosporin A and anti-TNF antibody.
